(19) United States
(12) Patent Application Publication
(10) Pub. No.: US 2011/0196393 A1
(43) Pub. Date: Aug. 11, 2011

(54) URETHRAL ANASTOMOSIS DEVICE AND METHOD OF USING THE SAME

(75) Inventors: Eliahu Eliachar, Haifa (IL); Nir Lilach, Kfar Yehoshua (IL); Dan Sade Hochstadter, Bet Alfa (IL); Ofer Yossepowitch, Petach Tikvah (IL)

(73) Assignee: KEREN MEDICAL LTD., Bet Alfa (IL)

(21) Appl. No.: 13/123,755

(22) PCT Filed: Oct. 20, 2009
(86) PCT No.: PCT/IL2009/000986

§ 371 Date: Apr. 26, 2011

Related U.S. Application Data
(60) Provisional application No. 61/106,619, filed on Oct. 20, 2008.

(51) Int. Cl. A61B 17/11; A61M 27/00

(52) U.S. Cl. 606/153; 604/544

(57) ABSTRACT

The present invention provides a urethral catheter 200 adapted for anastomosis following radical prostatectomy, comprising an inflated activating balloon 60 adapted for concurrently (i) pressing said-bladder neck 140 to saidurethra stub 120; (ii) effectively stretching suture 90; (iii) activating locks 80 that non-reversibly catch saidsutures 90; and (iv) cutting saiddistal portion thereof.

### FIELD OF THE INVENTION

The present invention relates to an instrument for anastomosis of two hollow organs, hollow organ and vessel or two vessels, and, more specifically, to a device for the anastomosis of the bladder neck and the urethral stump.

### BACKGROUND OF THE INVENTION

After certain operations are performed on a living body requiring the removal of certain body parts, other body parts must be reconnected in order for the patient to survive and maintain normal body functions. For example, in certain heart operations where bypass surgery is performed, sections of a person's coronary artery to the heart may be either completely replaced or actually bypassed during the heart bypass operation. While some of these arteries are large and can be easily manipulated by a surgeon, other arteries or hollow body organs might be smaller and more difficult to manipulate due to their location within the body.

In one instance, the prostate gland is being removed in an operation called a prostatectomy due to the occurrence of cancer in the prostate. After removal of the prostate, the retained urethral stump must be reconnected to the bladder in order for the person to resume normal body functions that is urination.

### SUMMARY OF THE INVENTION

It is hence one object of the invention to disclose a urethral catheter 200 adapted for anastomosis following radical prostatectomy, comprising an inflated activating balloon 60 adapted for concurrently (i) pressing the bladder neck 140 to the urethra stub 120; (ii) effectively stretching suture 90; (iii) activating locks 80 that non-reversibly catch the sutures 90; and (iv) cutting the distal portion thereof.

Another object of the invention to disclose a semi-rigid urethral catheter 200 as defined above, adapted for being consecutively inserted into patient's urethra 120 and urinary bladder 130 detached after radical prostatectomy; catheter 200 further comprising outer and inner members 10 and 20, respectively; said outer member 10 is provided at the side thereof with a special mark enabling a surgeon performing an anastomosis procedure under laparoscopic video control to insert the catheter 200 into the patient's urethra precisely, specifically, up to superimposing a rim of the patient's urethra and the aforesaid mark; said inner member 20 is provided at a distal end thereof with an anchoring balloon 100 covered when inserting/withdrawing the catheter 200 within patient's urinary tract by a cap 110; a needle assembly comprises a plurality of needles 30 disposed parallel to a generatrix of the catheter 200; said needle assembly is remotely controlled by the surgeon performing the anastomosis procedure; a plurality of U-shaped sutures 90 is also disposed parallel to generatrix of the catheter 200; each U-shaped suture has two ends: a first end of the suture 90 is releasably connected to a needle extremity 35; a second end of the suture 90 is connected to a non-reversible lock 80 releasably disposed at a sidely deployable activating means 70; wherein said needles 30, when inserted into the urethral stump, keep the position of the latter constat relative to the catheter 200, the course of the needles is predetermined by means selected from a group consisting of the shape of curved tunnel 37A; utilizing needles that characterized by shape memory properties or a combination of the two; wherein the anchoring balloon 100 is adapted to be inserted into the urinary bladder 130 through the bladder neck 140 and to partially inflate the anchoring balloon 100 within the bladder 130; in a manner that reciprocally actuatable shaft 110A is provided in its extended configuration; wherein the inflation of anchoring balloon 100 is provided by forcing a fluid throughout fluid inlet 160 at a given pressure, via conduit 50 positioned inside and along the main axis of catheter 200; wherein activating means 70 is adapted for being deployed in an approximate perpendicular manner, in respect to the main axis of the catheter, in a manner that balloon 100 is deflated, and shaft 110A is again in its elongated configuration; means 70 comprises an activating balloon 60 which is adapted to activate, inter alia, the non-reversible locks 80; wherein locks are adapted for non-reversibly locking the suture 90 when pressurized by the balloon 60; and wherein passage 40 is designed for delivering compressed air to the balloon 60; wherein needles 30 are configured so that they are advanced into the urinary bladder 120 through the non-reversible locks 80, possibly towards catchers 25 that disposed at the distal portion of the inner member 20; catchers 25 are further adapted to catch the sutures 90 delivered by the needles 30; and wherein said non-reversible locks 80 are adapted for pressure-induced activation due to inflation of the activating balloon 60 in a manner that when the balloon 60 is further inflated, it urges the urethra stub 120 and the bladder neck to conjoin while starching suture 90, the non-reversible locks 80 non-reversibly lock the sutures 90 and cut a distal portion of the sutures 90 roved through the non-reversible locks, and simultaneously the catchers 25 hold the proximal portions of the sutures 90 during cutting thereof.

Another object of the invention to disclose a urethral catheter 200 as defined above, comprising: a semi-rigid catheter body constituting a telescopic structure further comprising inner and outer members; said inner member is provided at a distal end thereof with an inflatable anchoring balloon adapted for fixating the bladder neck; said outer member includes a plurality of apertures on a side surface of said member thereof; a remotely controlled needle assemble comprising a plurality of needles; an extremity of each said needle is positioned at said corresponding aperture and adapted for directing outward through said aperture and an adjacent tissue; a plurality of U-shaped sutures having two ends; each first end thereof is releasably connected to an distal extremity of corresponding said needle; each said U-shaped suture is longitudinally placed in said outer member and adapted to be driven by corresponding said needle extremity through corresponding aperture into said adjacent tissue.

Another object of the invention to disclose a method of performing anastomosis following radical prostatectomy. The method comprises the steps of (a) obtaining urethral catheter having at least one inflatable activating balloon 60; and (b) concurrently (i) pressing the bladder neck 140 to the urethra stub 120; (ii) effectively stretching suture 90; (iii) activating locks 80 that non-reversibly catch the sutures 90; and (iv) cutting the distal portion thereof.

Another object of the invention to disclose method of performing anastomosis following radical prostatectomy comprising the steps of providing a urethral catheter 200 comprising a catheter body constituting a telescopic structure further comprising inner and outer members; said inner member is provided at a distal end thereof with an inflatable anchoring balloon adapted for fixating the bladder neck; said outer member includes a plurality of apertures on a side of said member thereof; a remotely controlled needle assemble comprising a plurality of needle; an extremity of each said needle is positioned at said corresponding aperture and adapted for directing outward through said aperture and an adjacent tissue; a plurality of U-shaped sutures having two ends; each first end thereof is releasably connected to an distal extremity of corresponding said needle; each said U-shaped suture is longitudinally placed in said outer member and adapted to be driven by corresponding said needle extremity through corresponding aperture into said adjacent tissue; each said U-shaped suture is provided at a second end thereof with a non-reversible lock; each said needle is configured for advancing through corresponding said non-reversible lock; each said non-reversible lock is adapted to catch and non-reversibly fixate said first end of corresponding said suture end when driven by corresponding said needle extremity, said outer member is provided at distal end thereof with activating means adapted for remotely activating said non-reversible locks; consecutively inserting said catheter into patient's urethra and urinary bladder; deploying said needles and sutures driven by said needles into patient's urethra stub; inflating said anchoring balloon; drawing together of patient's bladder neck and urethra stub by backwardly displacing said inner member carrying said balloon relative to said outer member; deflating said anchoring balloon; advancing said needles and sutures driven be said needles through patient's bladder wall and corresponding said non-reversible suture locks; withdrawing said needles from the patient's bladder; conjoining said bladder neck and urethra stub; activating said non-reversible locks; cutting off suture distal portions roved through said non-reversible locks; and withdrawing said catheter from the patient's urinary tract.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic cross-section view of the urethral catheter inserted into the urethra and the urinary bladder detached after radical prostatectomy;

FIG. 2 is an enlarged schematic view of the needle-suture arrangement;

FIG. 3 is a schematic view of the suture material;

FIG. 4 is a schematic cross-section view of the urethral catheter with the needle deployed into the urethra;

FIG. 5 is a schematic cross-section view of the urethral catheter with the inflated anchoring balloon;

FIG. 6 is a schematic cross-section view of the urethral catheter with the inflated anchoring balloon proximally displaced for drawing together the bladder neck and the urethra stump;

FIG. 7 is a schematic cross-section view of the urethral catheter with the deflated anchoring balloon and the deployed activating means;

FIG. 8 is a schematic cross-section view of the urethral catheter with the needles advanced through the non-reversible locks;

FIG. 9 is a schematic cross-section view of the urethral catheter with the needles withdrawn from the non-reversible locks;

FIG. 10 is a schematic cross-section view of the urethral catheter with the inflated activating balloon;

FIG. 11 is a schematic cross-section view of the urethral catheter with the inflated deflated balloon

FIG. 12 is a schematic cross-section view of the urethral catheter withdrawn from the patient's urinary tract; and

FIG. 13 is a flowchart of the method of performing anastomosis following radical prostatectomy.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a urethral catheter and a method of performing urethro-vesical anastomosis.

As used herein, the term “anastomosis” means the surgical connection of respective body lumens and other hollow body structures.

As used herein, the term “proximal” (or any form thereof), with respect to a component of an instrument, means that portion of the component that is generally nearest the surgeon, or nearest to the end of the instrument handled by the surgeon, when in use; and with respect to a direction of travel of a component of an instrument, means toward the end of the instrument generally nearest the surgeon, or handled by the surgeon, when in use.

As used herein, the term “distal” (or any form thereof), with respect to a component of an instrument, means that portion of the component that is generally farthest from the surgeon, or farthest from the end of the instrument handled by the surgeon, when in use; and with respect to a direction of travel of a component of an instrument, means away from the end of the instrument generally nearest the surgeon, or handled by the surgeon, when in use.

The term “generatrix” hereinafter refers to a geometric element that generates a geometric figure, especially a straight line that generates a surface by moving in a specified fashion.

The term “shape memory” hereinafter refers to the unique ability of shape memory alloys to be severely deformed and then returned to their original shape simply by heating them.

The term “pseudo-elasticity” hereinafter refers to a rubber-like flexibility demonstrated by shape memory alloys.

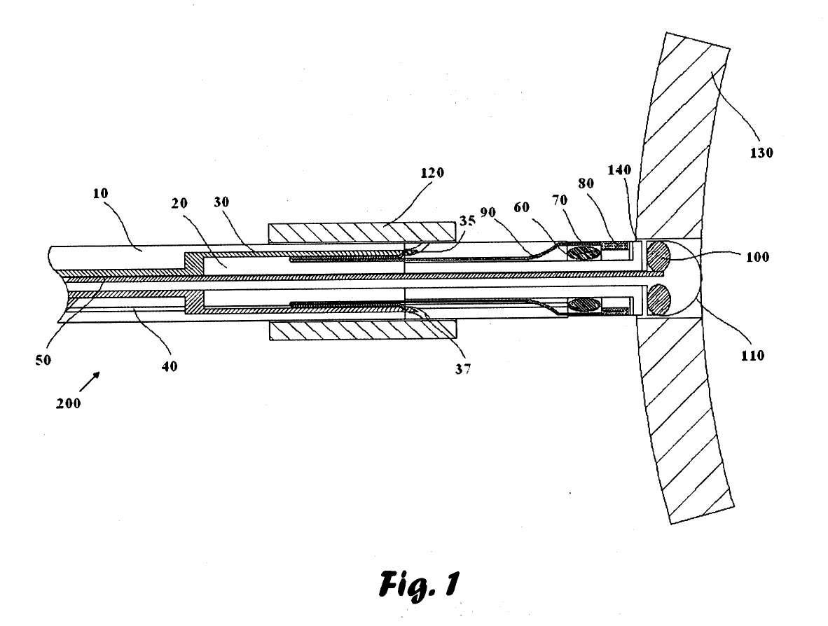

Reference is now made to FIG. 1, a schematic cross-section view of a semi-rigid urethral catheter 200 consecutively inserted into patient's urethra 120 and urinary bladder 130 detached after radical prostatectomy. The aforesaid catheter 200 comprises outer and inner members 10 and 20, respectively. The outer member 10 is provided at the side thereof with a special mark enabling a surgeon performing an anastomosis procedure under laparoscopic video control to insert the catheter 200 into the patient's urethra precisely, specifically, up to superimposing a rim of the patient's urethra and the aforesaid mark. The inner member 20 is provided at a distal end thereof with an anchoring balloon 100 covered when inserting/withdrawing the catheter 200 within patient's urinary tract by a cap 110. A needle assembly comprises a plurality of needles 30 disposed parallel to a generatrix of the catheter 200. The needle assembly is remotely controlled by the surgeon performing the anastomosis procedure. A plurality of U-shaped sutures 90 is also disposed parallel to generatrix of the catheter 200. Each U-shaped suture has two ends. A first end of the suture 90 is releasably connected to a needle extremity 35. A second end of the suture 90 is connected to a non-reversible lock 80 releasably disposed at a sidely deployable activating means 70.

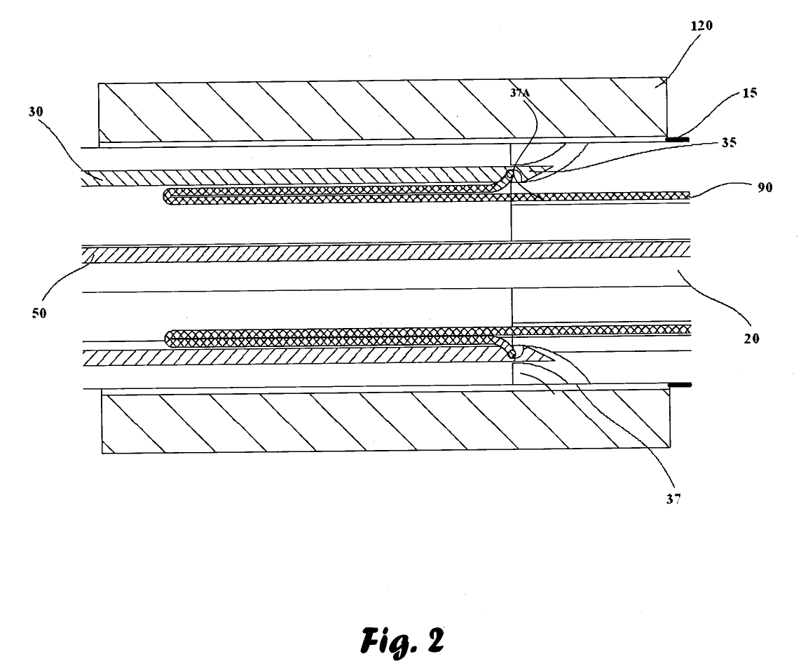

Reference is now made to FIG. 2, representing a scaled-up view of a needle-suture arrangement (cross section). The needles 30 are adapted to be deployed into the adjacent tissue of the urethral stump 120 through an aperture 37. As seen in FIG. 2, the suture 90 is drawn by the needle 30 into the patient's tissue 120 via curved tunnel 37A.

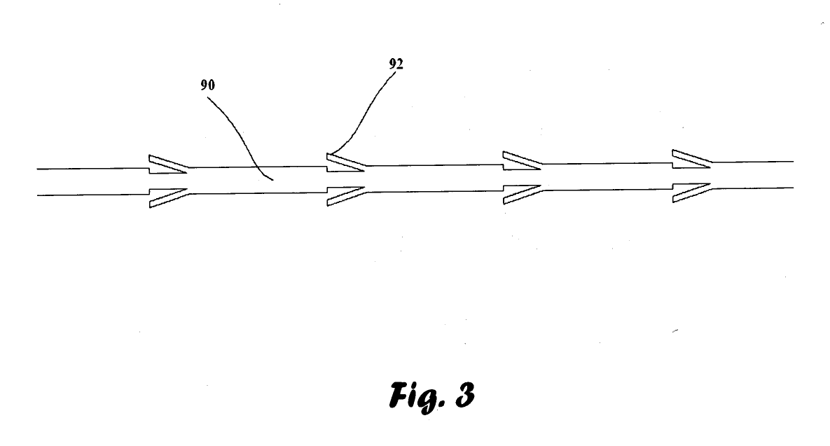

Reference is now made to FIG. 3, showing a configuration of the absorbable suture 90. The aforesaid suture 90 is furnished with projections 92 preventing backwardly displacing the suture 90 after inserting into the patient's tissues.

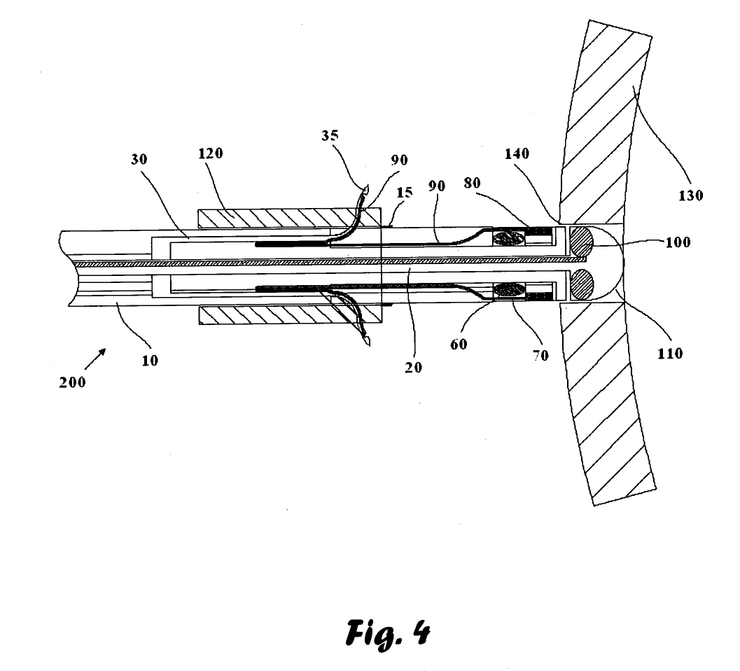

Reference is now made to FIG. 4, showing deployment of the tip of the needles 35 which interconnect with the sutures 90 into the urethral stump 120. The needles 30, when inserted into the urethral stump, keep the position of the latter constant relative to the catheter 200. The course of the needles is predetermined by means selected e.g., by the shape of curved tunnel 37A (see FIG. 2), by utilizing needles that characterized by shape memory properties or a combination of the two.

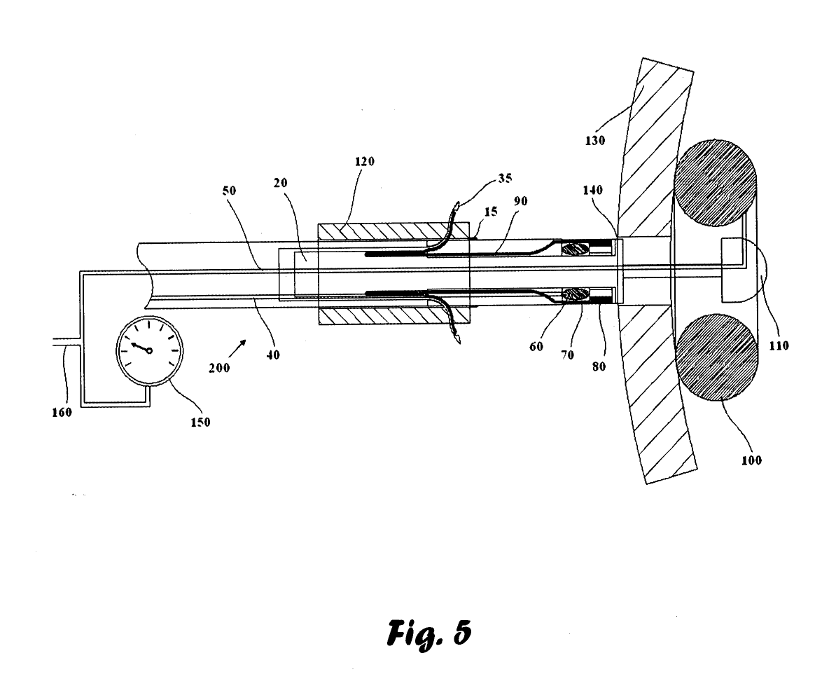

Reference is now made to FIG. 5, presenting inserting the anchoring balloon 100 into the urinary bladder 130 through the bladder neck 140 and partially inflating the anchoring balloon 100 within the bladder 130. Reciprocally actuatable shaft 110A is now in its extended configuration. In one embodiment of the invention, the inflation of anchoring balloon 100 is provided by forcing a fluid (e.g., air, saline etc) throughout fluid inlet 160 at a given pressure (See gauge 150), via conduit 50 positioned inside and along the main axis of catheter 200.

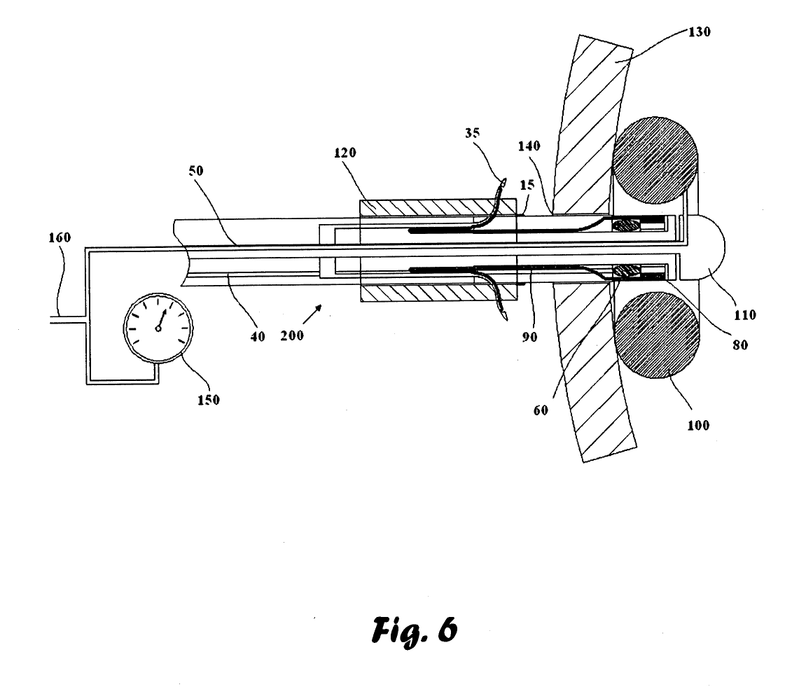

Reference is now made to FIG. 6, after inflation of balloon 100, bladder neck 140 is pulled by pulling shaft 110A to its shortened configuration, towards the urethra stub 120. The pressure of the fluid may increase as drawn in gauge 150, or remain unchanged.

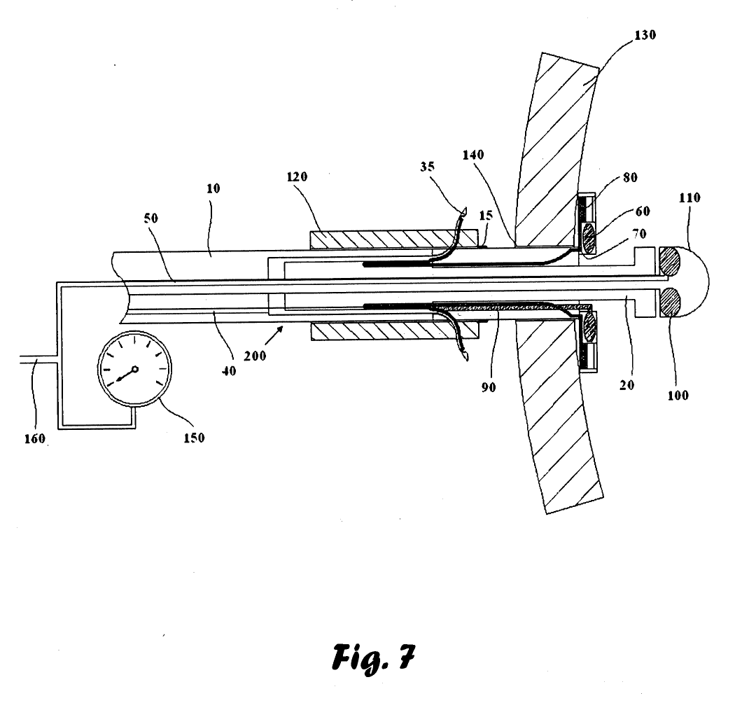

Reference is now made to FIG. 7, showing a view (cross section) of deployment of the activating means 70 in an approximate perpendicular manner, in respect to the main axis of the catheter. In this stage, balloon 100 is deflated (no pressure is presented by gauge 150), and shaft 110A is again in its elongated configuration. According to one embodiment of the invention, the aforesaid means 70 comprises an activating balloon 60 which is adapted to activate, inter alia, the non-reversible locks 80 (for example, crushable non-reversible locks). The locks are adapted for non-reversibly locking the suture 90 when pressurized by the balloon 60. The passage 40 is designed for delivering compressed air to the balloon 60.

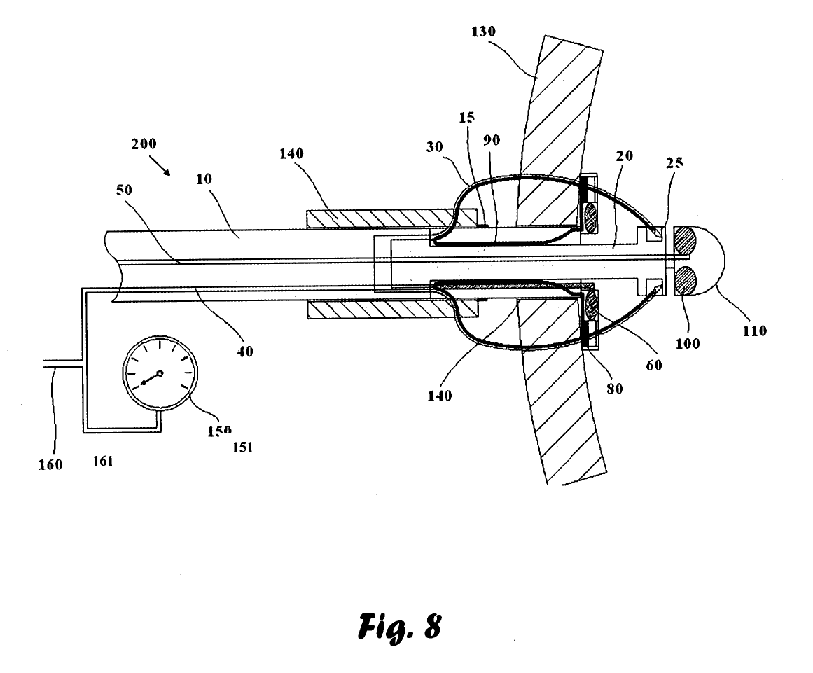

Reference is now made to FIG. 8, needles 30 are configured so that they are advanced into the urinary bladder 120 through the non-reversible locks 80, possibly towards catchers 25 that disposed at the distal portion of the inner member 20. The catchers 25 are adapted to catch the sutures 90 delivered by the needles 30. In accordance with one embodiment of the current invention, the needles 30 possess property of pseudo-elasticity. It means that a trajectory of the advanced needles 30 is curvilinear. The property of pseudo-elasticity enables the needles to create closed suture stitches. In accordance with another embodiment of the current invention, the non-reversible locks 80 are characterized as a net-like member, e.g., mesh, net or any other perforated or piercable matter of various sizes and shapes.

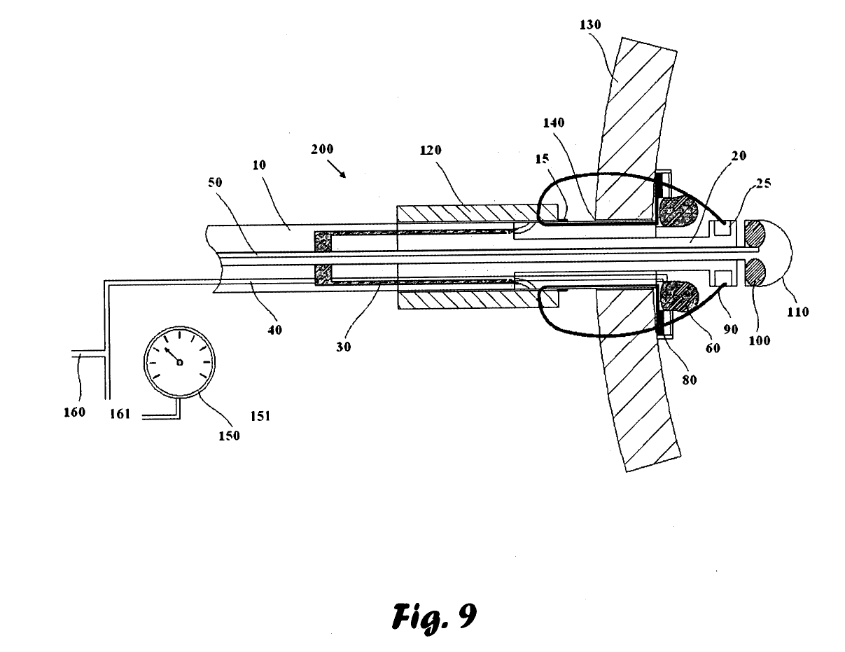

Reference is now made to FIG. 9, showing a cross section of the device, in the step of withdrawing the needles 30 from the patient's tissues, i.e., via both the urethra stub 120 and urinary bladder 130, throughout non-reversible locks 80 towards catchers 25. As shown in FIG. 3, the configuration of the suture 90 is adapted to prevent backward displacement of the suture 90 due to projections 92 (See FIG. 3). As gauge 151 shows, the activating balloon 60 is partially inflated by forcing a fluid (e.g., air, saline etc) throughout fluid inlet 161 at a suitable pressure.

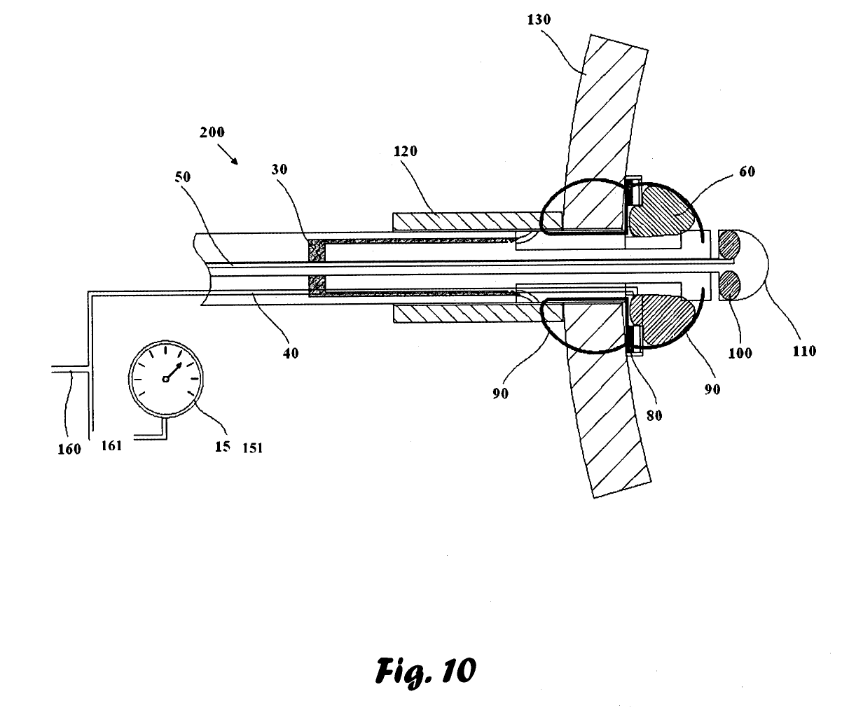

Reference is now made to FIG. 10, illustrating pressure-induced activating of the non-reversible locks 80 due to inflation of the activating balloon 60 (see high pressure displayed in gauge 151). When the balloon 60 is further inflated, it urges the urethra stub 120 and the bladder neck to conjoin while starching suture 90. Additionally, the non-reversible locks 80 are adapted for non-reversibly locking the sutures 90 and cutting a distal portion of the sutures 90 roved through the non-reversible locks. The catchers 25 (See in FIG. 10) hold the proximal portions of the sutures 90 during cutting thereof.

Thus, the inflated activating balloon 60 concurrently (i) presses the bladder neck 140 to the urethra stub 120; (ii) effectively stretches suture 90; (iii) activates locks 80 that non-reversibly catch the sutures 90; and finally (iv) cuts (e.g., by means of a razor-like, a guillotine-like or scissors-like sharp, possibly heated members) the distal portion thereof.

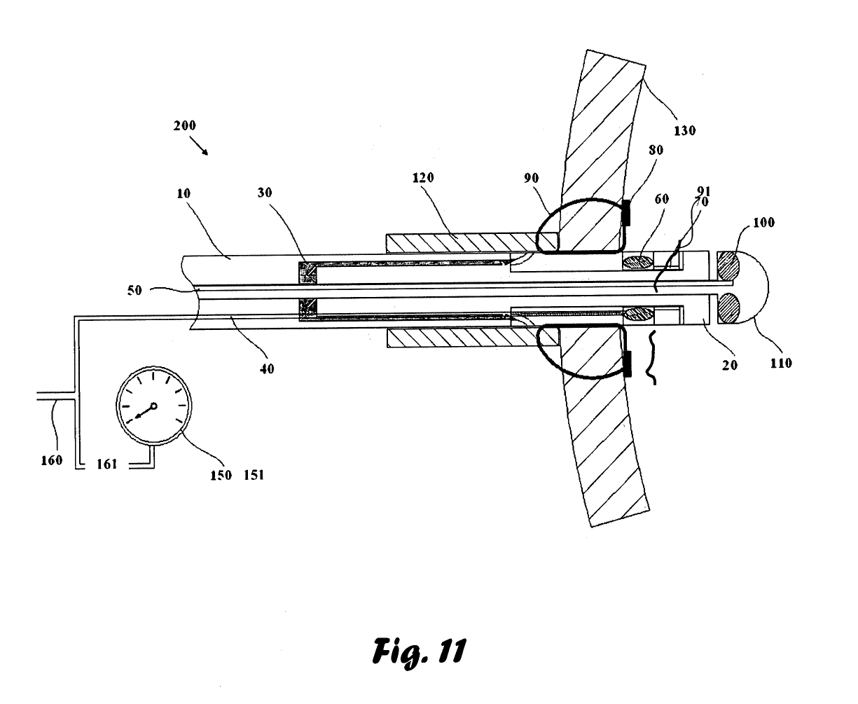

Reference is now made to FIG. 11, showing stitches created by the sutures 90 locked by the non-reversible locks 80; loose ends 91 of sutures 90 that were cut are also presented. The balloon 60 is deflated and activating means 70 is collapsed, see no pressure in gauge 151. Thus, the catheter 200 is ready for withdrawing. As said above, the sutures 90 are made of a biodegradable material and do not require further removal.

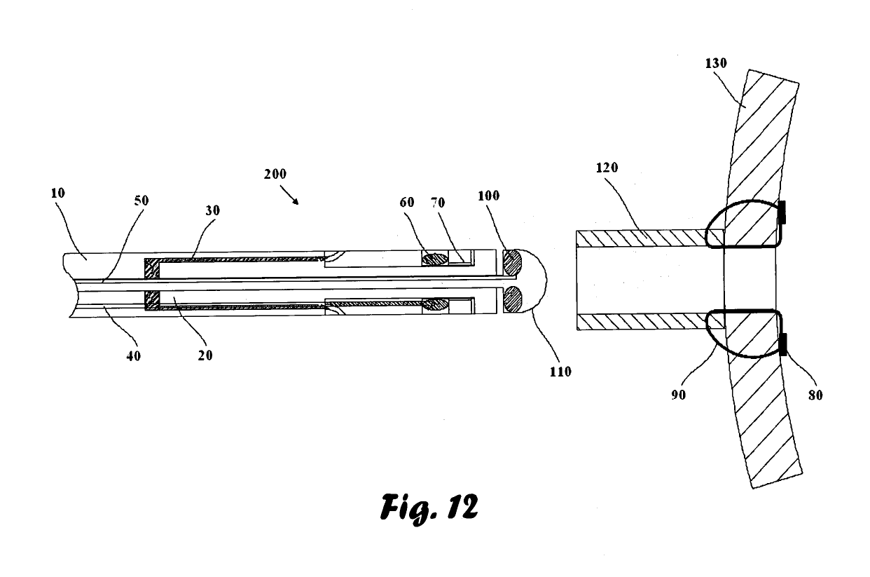

Reference is now made to FIG. 12, presenting the cross section of a urethral catheter 200 at the step it withdrawn from the patient's urinary tract. The urethra stub 120 and the bladder neck 140 detached during the radical prostatectomy procedure have conjoined by means of the urethral catheter 200.

Reference is now made to FIG. 13, showing a flowchart 300 of a method of performing anastomosis of the urethral stub and the bladder neck following radical prostatectomy. When provided at the step 310, the catheter is inserted into the patient's urinary tract, specifically, consecutively into the urethra and the urinary bladder detached after the radical prostatectomy procedure at the step 320. The urethra catheter is inserted into the urethra stub precisely, up to superimposing a rim of the urethra stub and the special mark at the outer member. The urethra stub is fixated relative to the catheter by means of deploying the needles and the sutures drawn by the aforesaid needles (the step 330). In order to draw the urethra stub and the bladder neck, the anchoring balloon is inflated and proximally displaced at the steps 340 and 350, respectively. Further, the anchoring balloon is deflated (the step 360). For performing the locked stitching, the needles with the sutures are advanced through the wall of the patient's urinary bladder and the non-reversible suture locks at the step 370. It should be emphasized that the sutures have the side projections preventing the backwardly displacing of the inserted sutures. Thus, at the step 380 the needles are withdrawn while the sutures are left in the patient's tissues. Further, the urethra stub and the bladder neck are completely conjoined due to gradually inflating the activating balloon. The aforesaid balloon activates the non-reversible suture locks adapted to cut the distal portions of the sutures. Specifically, at the step 390, the urethra stub and the bladder neck are conjoined. The locked stitches are created due to the balloon-assisted activating of the non-reversible locks and the suture distal portions are cut off at the steps 400 and 410, respectively. It is acknowledged in this respect that steps 390-410 comprises four actions provided simultaneously (step 401), whereas the inflated activating balloon 60 concurrently (i) presses the bladder neck 140 to the urethra stub 120; (ii) effectively stretches suture 90; (iii) activates locks 80 that non-reversibly catch the sutures 90; and finally (iv) cuts (e.g., by means of a razor-like, a guillotine-like or scissors-like sharp, possibly heated members) the distal portion thereof Finally, the catheter is withdrawn from the patient's urinary tract at the step 420.

In accordance with the current invention, the proposed urethral catheter is adapted for anastomosis following radical prostatectomy. Aforesaid catheter comprises a catheter body constituting a telescopic structure further comprising inner and outer members, a remotely controlled needle assemble comprising a plurality of needle and a plurality of U-shaped sutures. The inner member is provided at a distal end thereof with an inflatable anchoring balloon adapted for fixating the bladder neck. The outer member includes a plurality of apertures on a side of the member thereof. The extremity of each needle is positioned at the corresponding aperture and adapted for directing outward through the aperture and the adjacent tissue. Each suture has two ends. The first end thereof is releasably connected to a distal extremity of corresponding said needle. Each U-shaped suture is longitudinally placed in said outer member and adapted to be drawn by the corresponding needle extremity through the corresponding aperture into the adjacent tissue.

The main innovation is in the U-shaped sutures provided at second ends thereof with non-reversible locks. The needles are configured for advancing through the corresponding the non-reversible lock. The non-reversible locks are adapted to catch and non-reversibly fixate the first ends of the corresponding suture ends when driven by the corresponding needle extremities. The outer member is provided at distal end thereof with activating means adapted for remotely activating said non-reversible locks.

In accordance with the current invention, the activating means further comprises a plurality of sideways deployable members and an inflatable activating balloon. The non-reversible locks are disposed at the corresponding the deployable member. The activating balloon is further consecutively adapted to press the bladder neck to the urethra stub and activate locking the sutures by the non-reversible locks. The non-reversible lock is further adapted to cut a distal portion of the corresponding suture roved through said non-reversible lock.

In the accordance with one embodiment of the current invention, the non-reversible lock constitutes a mesh-like structure.

In the accordance with one embodiment of the current invention, the suture comprises sideways deployable members adapted to prevent proximal displacing said suture material.

In the accordance with one embodiment of the current invention, the outer member is provided at said side thereof with a distinguishable mark for disposing patient's urethra stub. The distinguishable mark is distinguishable by laparoscopic imaging means.

In the accordance with a further embodiment of the current invention, the inner member is provided at said distal portion thereof with a plurality of suture catchers adapted to catch extremities of said advanced sutures.

In the accordance with one embodiment of the current invention, needles constitute a muscle wire structure. The muscle wire structure is made of alloy chosen from the group consisting of Ag—Cd, Cu—Al—Ni, Cu—Sn, Cu—Zn, Fe—Pt, Mn—Cu, Fe—Mn—Si, Co—Ni—Al, Co—Ni—Ga, Ni—Fe—Ga, Ti—Pd, and Ni—Ti.

In the accordance with one embodiment of the current invention, the steps of conjoining said bladder neck and urethra stub and activating the non-reversible locks are performed by means inflating an activating balloon.

In the accordance with another embodiment of the current invention, the step of advancing the needles and the sutures driven by the needles further comprises fixating extremities of the sutures at said catchers.

In the accordance with another embodiment of the current invention, the step of inserting the catheter into patient's urethra is performed up to superimposing the mark at the outer member and patient's urethra stub. The anastomosis procedure is performed under laparoscopic video control.

1. A urethral catheter 200 adapted for anastomosis following radical prostatectomy, comprising an inflated activating balloon 60 adapted for concurrently (i) pressing saidbladder neck 140 to saidurethra stub 120; (ii) effectively stretching suture 90; (iii) activating locks 80 that non-reversibly catch saidsutures 90; and (iv) cutting saiddistal portion thereof.

2. A semi-rigid urethral catheter 200 as defined in claim 1, adapted for beingconsecutively inserted into patient's urethra 120 and urinary bladder 130 detached after radical prostatectomy; catheter 200 further comprising outer and inner members 10 and 20, respectively; said outer member 10 is provided at said side thereof with a special mark enabling a surgeon performing an anastomosis procedure under laparoscopic video control to insert said catheter 200 into said patient's urethra precisely, specifically, up to superimposing a rim of said patient's urethra and said aforesaid mark; said inner member 20 is provided at a distal end thereof with an anchoring balloon 100 covered when inserting/withdrawing said catheter 200 within patient's urinary tract by a cap 110; a needle assembly comprises a plurality of needles 30 disposed parallel to a generatrix of said catheter 200; said needle assembly is remotely controlled by said surgeon performing said anastomosis procedure; a plurality of U-shaped sutures 90 is also disposed parallel to generatrix of said catheter 200; each U-shaped suture has two ends: a first end of said suture 90 is releasably connected to a needle extremity 35; a second end of said suture 90 is connected to a non-reversible lock 80 releasably disposed at a sidely deployable activating means 70;
wherein said needles 30, when inserted into said urethral stump, keep said position of said latter constat relative to said catheter 200, said course of said needles is predetermined by means selected from a group consisting of said shape of curved tunnel 37A; utilizing needles that characterized by shape memory properties or a combination of said two;
wherein said anchoring balloon 100 is adapted to be inserted into said urinary bladder 130 through said bladder neck 140 and to partially inflate said anchoring balloon 100 within said bladder 130; in a manner that reciprocally actuatable shaft 110A is provided in its extended configuration;
wherein said inflation of anchoring balloon 100 is provided by forcing a fluid throughout fluid inlet 160 at a given pressure, via conduit 50 positioned inside and along said main axis of catheter 200;
wherein activating means 70 is adapted for being deployed in an approximate perpendicular manner, in respect to said main axis of said catheter, in a manner that balloon 100 is deflated, and shaft 110A is again in its elongated configuration; means 70 comprises an activating balloon 60 which is adapted to activate, inter alia, said non-reversible locks 80; wherein locks are adapted for non-reversibly locking said suture 90 when pressurized by said balloon 60; and wherein passage 40 is designed for delivering compressed air to said balloon 60;
wherein needles 30 are configured so that they are advanced into said urinary bladder 120 through said non-reversible locks 80, possibly towards catchers 25 that disposed at said distal portion of said inner member 20; catchers 25 are further adapted to catch said sutures 90 delivered by said needles 30;
and wherein said non-reversible locks 80 are adapted for pressure-induced activation due to inflation of said activating balloon 60 in a manner that when said balloon 60 is further inflated, it urges said urethra stub 120 and said bladder neck to conjoin while starching suture 90, said non-reversible locks 80 non-reversibly lock said sutures 90 and cut a distal portion of said sutures 90 roved through said non-reversible locks, and simultaneously said catchers 25 hold said proximal portions of said sutures 90 during cutting thereof.

3. The urethral catheter 200 as defined in claim 1, comprising:
a. a semi-rigid catheter body constituting a telescopic structure further comprising inner and outer members; said inner member is provided at a distal end thereof with an inflatable anchoring balloon adapted for fixating said bladder neck; said outer member includes a plurality of apertures on a side surface of said member thereof;
b. a remotely controlled needle assemble comprising a plurality of needles; an extremity of each said needle is positioned at said corresponding aperture and adapted for directing outward through said aperture and an adjacent tissue;
c. a plurality of U-shaped sutures having two ends; each first end thereof is releasably connected to an distal extremity of corresponding said needle; each said U-shaped suture is longitudinally placed in said outer member and adapted to be driven by corresponding said needle extremity through corresponding aperture into said adjacent tissue.

4. A method of performing anastomosis following radical prostatectomy comprising said steps of (a) obtaining urethral catheter having at least one inflatable activating balloon 60; and (b) concurrently (i) pressing said bladder neck 140 to said urethra stub 120; (ii) effectively stretching suture 90; (iii) activating locks 80 that non-reversibly catch said sutures 90; and (iv) cutting said distal portion thereof.

5. A method of performing anastomosis following radical prostatectomy comprising the steps of:
a. providing a urethral catheter 200 comprising
i. a catheter body constituting a telescopic structure further comprising inner and outer members; said inner member is provided at a distal end thereof with an inflatable anchoring balloon adapted for fixating said bladder neck; said outer member includes a plurality of apertures on a side of said member thereof;
ii. a remotely controlled needle assemble comprising a plurality of needle; an extremity of each said needle is positioned at said corresponding aperture and adapted for directing outward through said aperture and an adjacent tissue;
iii. a plurality of U-shaped sutures having two ends; each first end thereof is releasably connected to an distal extremity of corresponding said needle; each said U-shaped suture is longitudinally placed in said outer member and adapted to be driven by corresponding said needle extremity through corresponding aperture into, said adjacent tissue; each said U-shaped suture is provided at a second end thereof with a non-reversible lock; each said needle is configured for advancing through corresponding said non-reversible lock; each said non-reversible lock is adapted to catch and non-reversibly fixate said first end of corresponding said suture end when driven by corresponding said needle extremity, said outer member is provided at distal end thereof with activating means adapted for remotely activating said non-reversible locks;
b. consecutively inserting said catheter into patient's urethra and urinary bladder;
c. deploying said needles and sutures driven by said needles into patient's urethra stub;
d. inflating said anchoring balloon;
e. drawing together of patient's bladder neck and urethra stub by backwardly displacing said inner member carrying said balloon relative to said outer member;
f. deflating said anchoring balloon;
g. advancing said needles and sutures driven be said needles through patient's bladder wall and corresponding said non-reversible suture locks;
h. withdrawing said needles from said patient's bladder;
i. conjoining said bladder neck and urethra stub;
j. activating said non-reversible locks;
k. cutting off suture distal portions roved through said non-reversible locks; and
l. withdrawing said catheter from said patient's urinary tract.
